(19) United States
(12) Patent Application Publication
(10) Pub. No.: US 2018/0075204 A1
(43) Pub. Date: Mar. 15, 2018

(54) SYSTEMS AND METHODS FOR BUILDING SUPPORTIVE RELATIONSHIPS BETWEEN PATIENTS AND CAREGIVERS

(71) Applicant: KONINKLIJKE PHILIPS N.V., EINDHOVEN (NL)

(72) Inventors: Matthew Len LEE, New York, NY (US); Mladen MILOSEVIC, Stoneham, NY (US); Portia E. SINGH, Everett, NY (US)

(21) Appl. No.: 15/559,205

(22) PCT Filed: Mar. 16, 2016
(86) PCT No.: PCT/IB2016/051480

§ 371 Date: Sep. 18, 2017

Related U.S. Application Data
(60) Provisional application No. 62/135,201, filed on Mar. 19, 2015.

(51) Int. Cl. G06F 19/00

(52) U.S. Cl. CPC G06F 19/3418; G06Q 10/109; G06F 19/3406

(57) ABSTRACT

Systems and methods for providing prompts to improve communication between users and building supportive relationships are based on sharing activity information that characterizes one or more activities involving a patient and/or a caregiver among the patient and the caregiver. Moreover, the system determines and presents prompts for interactions between the patient and the caregiver based on the activity information.

### BACKGROUND

### 1. Field

The present disclosure pertains to a system and method for providing prompts to improve communication between patients and their caregivers, friends, and/or family, and, in particular, to share activity information among users that characterizes activities involving patients and their caregivers, friends, and/or family.

### 2. Description of the Related Art

It is common to gather information electronically from one or more information sources, including but not limited to medical devices that are carried and/or worn by patients. It is common to monitor patients and/or user others throughout the day, week, month, or year in order to gather information, including but not limited to information pertinent to caregivers and/or other users. It is common to process and/or analyze gathered information to extract pertinent information. It is common to present gathered and/or processed information to users through a user interface of a client computing platform.

### SUMMARY

Accordingly, one or more aspects of the present disclosure relate to a system configured to provide prompts to improve communication between users, the users including a subject and a caregiver. The system comprises physical storage configured to store information and one or more physical processors. The processors are configured via computer-readable instructions to obtain activity information characterizing one or more activities involving a subject, the activity information conveying one or both of a physical activity level for one or more time frames and/or calendar information for one or more time frames; generate an activity summary of the activity information; effectuate presentation of the activity summary on a user interface of a client computing platform that is associated with the caregiver; determine a prompt for an interaction between the subject and the caregiver, wherein the prompt is based on the activity information; and effectuate presentation of the prompt on the user interface of the client computing platform.

Yet another aspect of the present disclosure relates to a method of providing prompts to improve communication between users, the users including a subject and a caregiver. The method is implemented in a computer system that includes one or more physical processors and physical storage. The method comprises obtaining activity information characterizing one or more activities involving a subject, wherein the activity information conveys one or both of a physical activity level for one or more time frames and/or calendar information for one or more time frames; generating an activity summary of the activity information; effectuating presentation of the activity summary on a user interface of a client computing platform that is associated with the caregiver; determining a prompt for an interaction between the subject and the caregiver, wherein the prompt is based on the activity information; and effectuating presentation of the prompt on the user interface of the client computing platform.

Still another aspect of present disclosure relates to a system configured to provide prompts to improve communication between users, the users including a subject and a caregiver. The system comprises means for obtaining activity information characterizing one or more activities involving a subject, wherein the activity information conveys one or both of a physical activity level for one or more time frames and/or calendar information for one or more time frames; means for generating an activity summary of the activity information; means for effectuating presentation of the activity summary on a user interface of a client computing platform that is associated with the caregiver; means for determining a prompt for an interaction between the subject and the caregiver, wherein the prompt is based on the activity information; and means for effectuating presentation of the prompt on the user interface of the client computing platform.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 schematically illustrates a system configured to provide prompts to improve communications between users, in accordance with one or more embodiments;

FIG. 2 illustrates an exemplary user interface for a system configured to provide prompts to improve communications between users; and

FIG. 3 illustrates a method to provide prompts to improve communications between users, according to one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

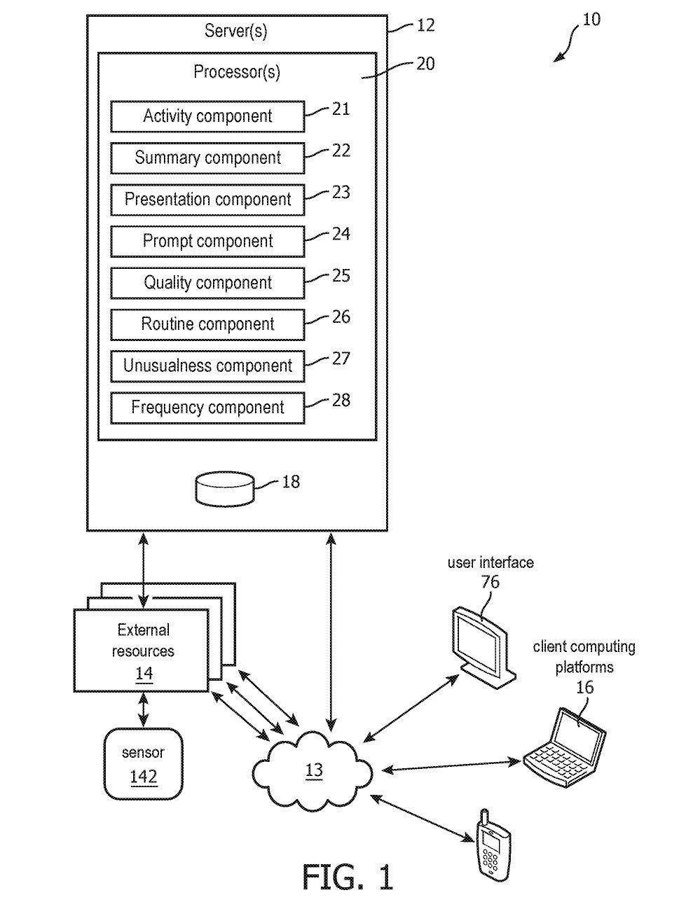

FIG. 1 illustrates a system 10 configured to provide prompts to improve communication between users, in accordance with one or more embodiments. Users may include subjects, patients, caregivers, friends, family members, and/or other users. For example, system 10 may be configured to provide prompts to a subject and/or a caregiver to improve the communication between the subject and the caregiver. In some embodiments, system 10 may be configured to share information regarding activities involving one user with another user. In some embodiments, system 10 may be configured to monitor, track, and/or quantify activity of one or more users. In some embodiments, system 10 may be configured to monitor, track, and/or quantify interactions between users. In some embodiments, system 10 may be configured to aid positive communications between users that support relationships and/or otherwise build affinity. For example, system 10 may be configured to share information regarding activities of a particular user living alone (or mostly alone) with a family member who cannot be physically present with the particular user every day. By sharing information and providing prompts as described in this disclosure, the interactions between the particular user and the family member may be improved (e.g. more frequent, more positive, more reciprocal, more engaging, more supportive, and/or in any other way improved). In some embodiments, system 10 may be configured to facilitate communication between users.

System 10 may include one or more servers 12, one or more physical processors 20, various computer program components, one or more client computing platforms 16, electronic storage 18, one or more external resources 14, and/or other components. The computer program components may include an activity component 21, a summary component 22, a presentation component 23, a prompt component 24, a quality component 25, a routine component 26, an unusualness component 27, a frequency component 28, and/or other components.

As is shown in FIG. 1, system 10 may operate in communication and/or coordination with one or more external resources 14. Users may interface with system 10 and/or external resources 14 via client computing platforms 16. The components of system 10 may be operatively linked via one or more electronic communication links. For example, such electronic communication links may be established, at least in part, via a network 13 such as the Internet and/or other (communication) networks. It will be appreciated that this is not intended to be limiting, and that the scope of this disclosure includes embodiments in which components may be operatively linked via some other communication media.

A given client computing platform 16 may include one or more processors configured to execute computer program components. The computer program components may be configured to enable one or more users associated with the given client computing platform 16 to interface with system 10 and/or external resources 14, and/or provide other functionality attributed herein to client computing platforms 16. By way of non-limiting example, the given client computing platform 16 may include one or more of a desktop computer, a laptop computer, a tablet computing device, a handheld computer, a NetBook, a smartphone, a gaming console, an interactive television, and/or other computing platform or computing device. In some embodiments, client computing platforms 16 may be configured to facilitate communication between users, e.g. through user interface structures. For example, a caregiver may communicate with a subject through a user interface structure of a client computing platform 16 that is associated with the caregiver. The subject may, e.g. in response, communicate with the caregiver through another user interface structure of another client computing platform 16 that is associated with the subject.

External resources 14 may include sources of activity information and/or other information (e.g. 3^{rd} party information sources, websites, medical devices, fitness devices, pedometers, activity trackers, personal devices that may be carried on a subject and/or worn by a subject, household appliances, consumer electronics, televisions, telephones, etc.), external entities participating with system 10, and/or other resources. In some embodiments, some or all of the functionality attributed herein to external resources 14 may be provided by resources included in system 10. For example, an external resource 14 may include a watch configured to measure and/or monitor one or more physiological characteristics of a subject. In some embodiments, external resource 14 may include one or more sensors 142.

One or more sensors 142 of system 10 in FIG. 1 may be configured to generate output signals conveying information related to physiological, environmental, and/or subject-specific (medical) parameters, and/or other information. System 10 may use any of the generated output signals to monitor the subject. In some embodiments, the conveyed information may be related to parameters associated with the state and/or condition of the subject, motion of the subject, wakefulness and/or sleep state of the subject, the breathing of the subject, the heart rate of the subject, the respiratory rate of the subject, vital signs of the subject, including one or more temperatures, oxygen saturation of arterial blood (SpO₂), whether peripheral or central, and/or other parameters.

In some embodiments, one or more sensors 142 may generate output signals conveying information related to a position and/or location of the subject, e.g. through stereoscopy, global positioning system (GPS) information, and/or other information. The location may be a three-dimensional location of the subject, a two-dimensional location of the subject, a location of a specific body part of the subject (e.g., eyes, arms, legs, a face, a head, a forehead, and/or other anatomical parts of the subject), the posture of the subject, the orientation of the subject or one or more anatomical parts of the subject, and/or other locations.

In some embodiments, one or more sensors 142 may generate output signals conveying information related to the subject's speech and/or interaction with other users. For example, a particular external resource 14 may monitor, track, and/or quantify interactions between users.

Sensors 142 may include one or more of a light sensor, an optical sensor, a temperature sensor, a pressure sensor, a weight sensor, an electromagnetic (EM) sensor, an infra-red (IR) sensor, a microphone, a transducer, a still-image camera, a video camera, and/or other sensors and combinations thereof.

The illustration of sensor 142 including one member in FIG. 1 is not intended to be limiting. System 10 may include one or more sensors. The illustration of a particular symbol or icon for sensor 142 in FIG. 1 is exemplary and not intended to be limiting in any way. Resulting signals or information from one or more sensors 142 may be transmitted to processor 20, user interface structure 76, electronic storage 18, and/or other components of system 10. This transmission can be wired and/or wireless.

One or more sensors 142 may be configured to generate output signals in an ongoing manner, e.g. throughout the day, week, month, and/or year(s). This may include generating signals intermittently, periodically (e.g. at a sampling rate), continuously, continually, at varying intervals, and/or in other ways that are ongoing during at least a portion of period of a day, week, month, or other duration. The sampling rate may be about 0.001 second, 0.01 second, 0.1 second, 1 second, about 10 seconds, about 1 minute, and/or other sampling rates. It is noted that multiple individual sensors may operate using different sampling rates, as appropriate for the particular output signals and/or (frequencies related to particular) parameters derived therefrom. For example, in some embodiments, the generated output signals may be considered as a vector of output signals, such that a vector includes multiple samples of information conveyed related to one or more parameters of the subject. Different parameters may be related to different vectors. A particular parameter determined in an ongoing manner from a vector of output signals may be considered as a vector of that particular parameter.

Server 12 may be configured to provide prompts to improve communication between users. Server 12 may be configured to interact with client computing platforms 16 to provide prompts to improve communication between users. Operation by server 12 may include hosting, serving, and/or otherwise providing a system within which interaction between users is facilitated via client computing platforms 16. Server 12 may include electronic storage 18, one or more processors 20, and/or other components. Server 12 may include communication lines, or ports to enable the exchange of information with a network and/or other computing platforms.

Physical processor 20 (interchangeably referred to herein as processor 20) is configured to provide information processing and/or system control capabilities in system 10. As such, processor 20 may include one or more of a digital processor, an analog processor, a digital circuit designed to process information, an analog circuit designed to process information, and/or other mechanisms for electronically processing information. In order to provide the functionality attributed to processor 20 herein, processor 20 may execute one or more components. The one or more components may be implemented in software; hardware; firmware; some combination of software, hardware, and/or firmware; and/or otherwise implemented. Although processor 20 is shown in FIG. 1 as a single entity, this is for illustrative purposes only. In some embodiments, processor 20 may include a plurality of processing units. These processing units may be physically located within the same device, or processor 20 may represent processing functionality of a plurality of devices operating in coordination (e.g., “in the cloud”, and/or other virtualized processing solutions).

As is shown in FIG. 1, processor 20 is configured to execute one or more computer program components. The one or more computer program components include one or more of activity component 21, summary component 22, presentation component 23, prompt component 24, quality component 25, routine component 26, unusualness component 27, frequency component 28, and/or other components. Processor 20 may be configured to execute components 21-28 by software; hardware; firmware; some combination of software, hardware, and/or firmware; and/or other mechanisms for configuring processing capabilities on processor 20.

It should be appreciated that although components 21-28 are illustrated in FIG. 1 as being co-located within a single processing unit, in embodiments in which processor 20 includes multiple processing units, one or more of components 21-28 may be located remotely from the other components. The description of the functionality provided by the different components 21-28 described below is for illustrative purposes, and is not intended to be limiting, as any of components 21-28 may provide more or less functionality than is described. For example, one or more of components 21-28 may be eliminated, and some or all of its functionality may be provided by other ones of components 21-28. Note that processor 20 may be configured to execute one or more additional components that may perform some or all of the functionality attributed below to one of components 21-28. It will be appreciated that the illustration of modules 21-28 being executed solely on processor 20 separate from client computing platforms 16 and external resources 14 is not intended to be limiting. For example, in some implementations, the client computing platforms 16 may be configured to provide locally at least some of the functionality attributed above to one or more of modules 21-28 Similarly, one or more of modules 21-28 may be executed locally on individual client computing platforms 16 while others are executed on server 12.

As used herein, the term “determine” (and derivatives thereof) may include measure, calculate, compute, estimate, approximate, generate, and/or otherwise derive, and/or any combination thereof. As used herein, the term “obtain” (and derivatives thereof) may include active and/or passive retrieval, determination, derivation, transfer, upload, download, submission, and/or exchange of information, and/or any combination thereof.

Activity component 21 may be configured to obtain and/or determine activity information related to users. Activity information may characterize activities involving users. In some embodiments, activity information conveys one or both of a physical activity information and/or calendar information. In some embodiments, physical activity information may include a physical activity level, information regarding actions performed by users, and/or other information. For example, information regarding actions performed by an individual user may include information about a meal the user has cooked (for example at which time, and consisting of which kinds of food), coffee that has been made, television that has been watched (for example at which time, and consisting of which programs or shows), medications that have been taken, websites that have been visited, and/or other actions performed by the user. Alternatively, and/or simultaneously, in some embodiments, activity information may include geographical information and/or location information regarding present and/or past locations of users. Alternatively, and/or simultaneously, in some embodiments, activity information may be associated with one or more time frames. For example, a particular physical activity level may be associated with a particular time frame. For example, information from a pedometer may be activity information for a particular hour, day, week, and/or other time frame. For example, calendar information may be obtained from a client computing platform that is associated with a particular user, including but not limited to a particular user's smartphone. In some embodiments, activity information may be obtained from one or more external resources 14 and/or one or more sensors 142. In some embodiments, a particular type of activity information may be directly retrieved from a particular external resource 14. Alternatively, and/or simultaneously, in some embodiments, a particular type of activity information may be inferred from other information, for example through a particular external resource.

In some embodiments, activity component 21 may be configured to determine parameters from output signals generated by one or more sensors 142. Parameters may be related to a user's physiological, environmental, and/or user-specific parameters. For example, parameters may be related to monitored vital signs of the subject, parameters related to whether the subject is awake or asleep, the air temperature, ambient noise level, ambient light level, and/or other parameters. In some embodiments, activity information may be related to a user's interactions with other users.

In some embodiments, activity component 21 may be configured to determine, track, and/or monitor activity information and/or one or more parameters during a time frame spanning minutes, hours, days, and/or weeks. For example, in some embodiments, activity component 21 may be configured to determine a activity parameter, based on output signals generated by one or more sensors 142, during a time frame spanning at least a day, and/or intermittently, periodically (e.g. at a sampling rate), continuously, continually, at varying intervals, and/or in other ways that are ongoing during at least a period of a day, week, month, or other duration. For example, activity component 21 may be configured to determine a vector of activity parameters.

Activity information and/or parameters derived therefrom may be electronically stored in electronic storage 18. In some embodiments, information stored in electronic storage 18 may include a database.

Summary component 22 may be configured to generate and/or determine an activity summary of a particular set of activity information. In some embodiments, summary component 22 may generate an activity summary for a particular time frame, including but not limited to the present day, the current week, the elapsed time since a particular event, and/or other time frames. For example, the particular event may be the previous occasion that a particular user interacted or otherwise communicated with a subject. Information in a particular set of activity information may be compressed, analyzed, filtered, organized, grouped, and/or otherwise processed during generation of an activity summary. For example, an activity summary may include less information than the particular set of activity information that it is based on. In some embodiments, information in the particular set of activity information that is not pertinent and/or less important for the purposes described in this disclosure may be omitted from an activity summary. In some embodiments, summary component 22 may be configured to learn, e.g. via user feedback, which types of information should or should not be included in an activity summary. In some embodiments, an activity summary may be presented as a timeline of events. In some embodiments, a user may adjust the particular time frame of a particular activity summary, for example by adjusting the beginning time, the ending time, the duration, and/or any combination thereof.

Presentation component 23 may be configured to effectuate presentation of information on client computing platforms 16. A particular client computing platform 16 may be associated with a particular user. For example, presentation component 23 may be configured to effectuate presentation of information, including but not limited to an activity summary, on user interface structure 76 of a particular client computing platform 16. In some embodiments, presentation component 23 may be configured to effectuate presentation of prompts (e.g. as determined by prompt component 24) on client computing platforms 16.

Prompt component 24 may be configured to determine prompts for interaction between users. In some embodiments, prompts may include one or more of descriptions of topics for conversations, activities or games, books, shows, or movies to share, and/or other suggestions that may involve interaction between users, including but not limited to written and/or verbal communication. In some embodiments, prompts may include textual information. Alternatively, and/or simultaneously, in some embodiments, prompts may include audio information. Alternatively, and/or simultaneously, in some embodiments, prompts may include video information. Prompts may be intended to improve communication between users. In some embodiments, prompts may be based on activity information, activity summaries, and/or other information. In some embodiments, prompts may be further based on information from other computer program components. For example, a particular prompt may include textual information that suggests to a caregiver to discuss a particular topic with a subject. For example, the particular topic may be related to recent activity information, such as a meeting with a friend, a stroll in the park, an unusual calendar entry, and/or other recent activity information. For example, a particular prompt may include information related to the timing and/or frequency of interaction between a caregiver and a subject. For example, the particular prompt may remind the caregiver that the subject has not yet called or send a text message today, whereas that is a common activity on, say, Mondays, or afternoons, or after particular activities.

Quality component 25 may be configured to determine quality metrics that reflect the quality of a set of interactions or conversation between users. Quality metrics may be based on obtained activity information, activity summaries, and/or other information. In some embodiments, a quality metric may reflect a level of reciprocity of a set of interactions between two particular users. For example, system 10 may be configured to monitor an exchange of text messages between two users. If text messages in a set of text messages alternate between the two users, the level of reciprocity may be higher than if many or most text messages in the set of text messages originate from the same user. Alternatively, and/or simultaneously, a quality level may reflect a frequency of interactions between two particular users. Alternatively, and/or simultaneously, a quality metric may reflect a level of positivity of a set of interactions between two particular users.

In some embodiments, system 10 may be configured to analyze the substance of individual text messages in a set of text messages. Based on the tone of a text message and/or the particular words used, an individual text message and/or a set of text messages may be associated with a high, low, and/or another level of positivity. The description of text messages regarding the determination of quality metrics is merely exemplary and not intended to be limiting in any way. Quality metrics may represent numerical values, percentages, grades, degrees, and/or combinations thereof. Prompts determined by prompt component 24 may be further based on one or more quality metrics. For example, a particular prompt may include textual information that suggests to a caregiver to improve the reciprocity of interaction and/or that suggests a particular topic or activity designed to improve the reciprocity of interaction. For example, a particular prompt may include textual information that suggests to a caregiver to improve the frequency of interaction and/or that suggests a particular topic or activity designed to improve the frequency of interaction. For example, a particular prompt may include textual information that suggests to a caregiver to improve the positivity of interaction and/or that suggests a particular topic or activity designed to improve the positivity of interaction.

Routine component 26 may be configured to determine a common set of activities for a particular user, and/or for a set of users. The set of activities may reflect an average day, an average week, and/or another time frame for a particular user, and/or for a set of users. In some embodiments, routine component 26 may be configured to determine routine activities for a user. In some embodiments, users may provide guidance to routine component 26, e.g. through input via a user interface, regarding particular activities and/or particular activity information, and whether these should or should not be considered as routine activities. For example, routine activities may be less likely than non-routine activities to be included in an activity summary and/or be used in determining a prompt for interaction between users. For example, an after-lunch walk in the park on the same weekdays as in the previous few weeks may be considered a routine activity.

Unusualness component 27 may be configured to determine whether particular activities (and/or the lack of particular routine activities) are unusual for a particular user, and/or for a set of users. Determinations of unusualness may be based on one or more comparisons with routine activities, e.g. a common set of activities as determine by routine component 26. Prompts determined by prompt component 24 may be further based on one or more determinations by unusualness component 27. In some embodiments, users may provide guidance to unusualness component 27, e.g. through input via a user interface, regarding particular activities and/or particular activity information, and whether these should or should not be considered as unusual activities. For example, unusual activities may be more likely than other activities to be included in an activity summary and/or be used in determining a prompt for interaction between users. For example, the lack of an after-lunch walk in the park on a particular weekday may be considered unusual if similar walks occurred in the previous few weeks. For example, a much shorter or longer walk in the park (compared to other walks in the park) may be considered unusual. For example, a walk in the park at an unusual time of day (compared to other walks in the park) may be considered unusual. For example, a walk in a different location than unusual (compared to other walks) may be considered unusual.

Frequency component 28 may be configured to determine rates of interactions between users. In some embodiments, frequency component 28 may be configured to establish a baseline of interaction between two particular users. Determinations by frequency component 28 may be associated with one or more particular time frames. For example, frequency component 28 may be configured to determine a first rate of interactions between two particular users during a first time frame. For example, the first time frame may span at least a week. The first rate of interactions may function as a baseline for how often two users typically or commonly interact. Frequency component 28 may be configured to determine a second rate of interactions between the two particular users during a second time frame. For example, the second time frame may be (at least on average) more recent than the first time frame. For example, the second time frame may be the most recent day, 24 hours, 48 hours, week, and/or other time frame. For example, the first time frame may span more time than the second time frame, e.g. the past week, month, or year-to-date. Frequency component 28 may be configured to determine a different between rates of interactions. For example, frequency component 28 may be configured to determine a difference between the first rate and the second rate. Frequency component 28 may be configured to compare the difference with one or more thresholds. For example, breaching a differential rate threshold may indicate a sudden and/or pertinent increase or decrease in the rate of interactions between the two particular users. Frequency component 28 may be configured to detect and/or determine a deviation from a baseline of interactions between two particular users. Prompts determined by prompt component 24 may be further based on one or more determinations by frequency component 28.

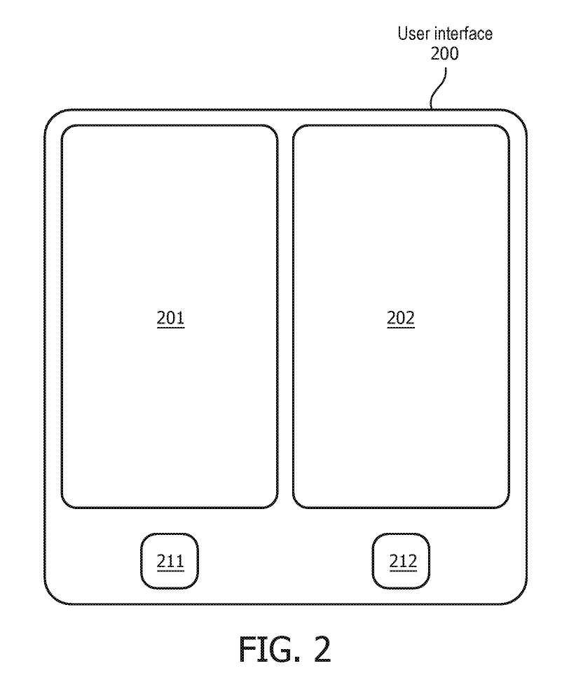

By way of illustration, FIG. 2 illustrates a view 200 of a graphical user interface 201 (interchangeably referred to as user interface 201) as may be presented to a user, e.g. a caregiver, to provide an activity summary related to a particular subject and/or one or more prompts for interaction with the particular subject. User interface 201 may be presented through a client device such as client computing platform 16 (depicted in FIG. 1). User interface 201 may be presented in response to the user interacting with system 10. Elements of user interface 201 may be implemented as fields configured to receive entry, selection, and/or confirmation from, e.g., the caregiver. The fields may include one or more of a text entry field, a set of selectable menu items, a selectable field, and/or other fields configured to receive entry, selection, confirmation from a user, and/or user interaction. The number of elements, components, and/or (user-selectable) fields depicted in FIG. 2 is meant to be exemplary, and not limiting in any way. User interface 201 may be configured to present information to and/or enable interaction with a user.

User interface 201 of FIG. 2 may include interface elements 211 and 212, informational element 201 and 202, and/or other components or features. Interface elements 211 and 212 may be, e.g., an object, interface, and/or other items that a user may interact with, such as a menu of user-selectable options and/or actions for engaging the system.

For example, interface element 211 may display information that represents a timeframe, including but not limited to “today,” “this week,” “the past few days,” and/or other timeframes. In some embodiments, interface element 211 may be adjustable and/or changeable by the user. Interface element 201 may display activity information for the particular subject. For example, the displayed information may include an activity summary of the particular subject for the particular time frame that corresponds to interface element 211. Interface element 202 may display one or more prompts (e.g. as determined by prompt component 24 in FIG. 1) for an interaction between the user and the particular subject.

For example, interface element 212 may support entry, selection, and/or confirmation from the user, e.g. related to a particular activity selected through interface element 201, a particular prompt selected through interface element 202, and/or related to other information that is pertinent to the operation of the system described in this disclosure. For example, the user may use interface element 212 to provide feedback and/or input about which activities should or should not be considered routine or unusual. For example, the user may use interface element 212 to provide feedback regarding a particular prompt. In some embodiments, a user interface similar to user interface 201 may support and/or facilitate the exchange of communication between users. For example, the user may communicate with the particular subject, including but not limited to textual messages, audio information, video information, and/or other information.

User interface structure 76 is configured to provide an interface between system 10 and a user through which the user can provide and/or receive information. In some embodiments, user interface structure 76 may be configured to facilitate the exchange of communication between users, including but not limited to textual messages, audio information, video information, and/or other information. This enables data, results, and/or instructions and any other communicable items, collectively referred to as “information,” to be communicated between the user and system 10. Examples of interface devices suitable for inclusion in user interface structure 76 include a keypad, buttons, switches, a keyboard, knobs, levers, a display screen, a touch screen, speakers, a microphone, an indicator light, an audible alarm, and a printer. Information may be provided to the subject by user interface structure 76 in the form of auditory signals, visual signals, tactile signals, and/or other sensory signals.

By way of non-limiting example, user interface structure 76 may include a light source capable of emitting light. The light source may include, for example, one or more of at least one LED, at least one light bulb, a display screen, and/or other sources. User interface structure 76 may control the light source to emit light in a manner that conveys to the subject information related to operation of system 10. Note that subject 106 and the user of system 10 may be one and the same person.

It is to be understood that other communication techniques, either hard-wired or wireless, are also contemplated herein as user interface structure 76. For example, in one embodiment, user interface structure 76 may be integrated with a removable storage interface provided by electronic storage 18. In this example, information is loaded into system 10 from removable storage (e.g., a smart card, a flash drive, a removable disk, etc.) that enables the user(s) to customize the implementation of system 10. Other exemplary input devices and techniques adapted for use with system 10 as user interface structure 76 include, but are not limited to, an RS-232 port, RF link, an IR link, modem (telephone, cable, Ethernet, internet or other). In short, any technique for communicating information with system 10 is contemplated as user interface structure 76.

Electronic storage 18 of system 10 in FIG. 1 comprises physical electronic storage media that electronically stores information, e.g. digital information. The electronic storage media of electronic storage 18 may include one or both of system storage that is provided integrally (i.e., substantially non-removable) with system 10 and/or removable storage that is removably connectable to system 10 via, for example, a port (e.g., a USB port, a FireWire port, etc.) or a drive (e.g., a disk drive, etc.). Electronic storage 18 may include one or more of optically readable storage media (e.g., optical disks, etc.), magnetically readable storage media (e.g., magnetic tape, magnetic hard drive, floppy drive, etc.), electrical charge-based storage media (e.g., EPROM, EEPROM, RAM, etc.), solid-state storage media (e.g., flash drive, etc.), network-attached storage (NAS), and/or other electronically readable storage media. Electronic storage 18 may include virtual storage resources, such as storage resources provided via a cloud and/or a virtual private network. Electronic storage 18 may store software algorithms, information determined by processor 20, information received via user interface 76, and/or other information that enables system 10 to function properly. For example, electronic storage 18 may record or store obtained activity information (as discussed elsewhere herein), and/or other information. The illustration of electronic storage 18 in FIG. 1 as being included in server 12 is exemplary and not intended to be limiting in any way. Electronic storage 18 may be a separate component within system 10, or electronic storage 18 may be provided integrally with one or more other components of system 10 (e.g., processor 20).

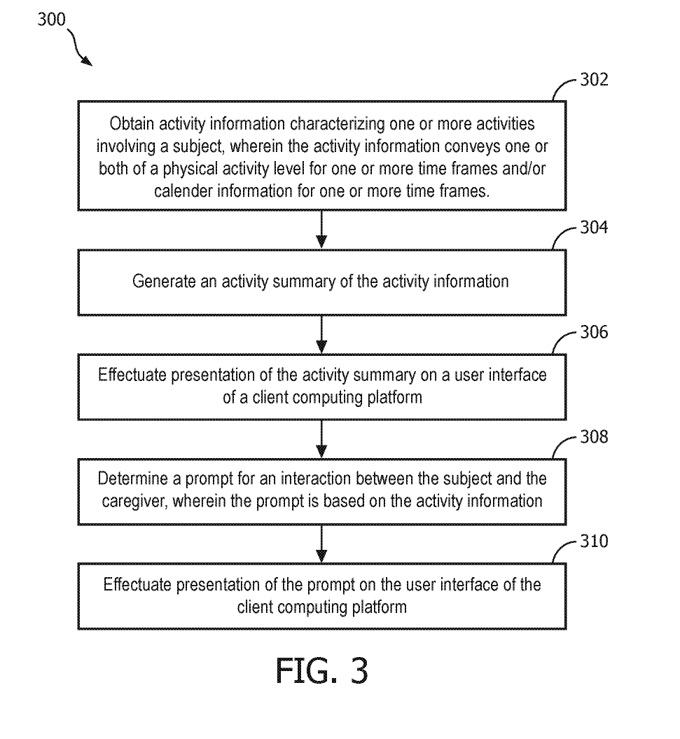

FIG. 3 illustrates a method 300 for providing prompts to improve communication between users. The operations of method 300 presented below are intended to be illustrative. In some embodiments, method 300 may be accomplished with one or more additional operations not described, and/or without one or more of the operations discussed. Additionally, the order in which the operations of method 300 are illustrated in FIG. 3 and described below is not intended to be limiting.

In some embodiments, method 300 may be implemented in one or more processing devices (e.g., a digital processor, an analog processor, a digital circuit designed to process information, an analog circuit designed to process information, and/or other mechanisms for electronically processing information). The one or more processing devices may include one or more devices executing some or all of the operations of method 300 in response to instructions stored electronically on an electronic storage medium. The one or more processing devices may include one or more devices configured through hardware, firmware, and/or software to be specifically designed for execution of one or more of the operations of method 300.

At an operation 302, activity information is obtained that characterizes one or more activities involving a subject. The activity information conveys one or both of a physical activity level for one or more time frames and/or calendar information for one or more time frames. In some embodiments, operation 302 is performed by an activity component the same as or similar to activity component 21 (shown in FIG. 1 and described herein).

At an operation 304, an activity summary of the activity information is generated. In some embodiments, operation 304 is performed by a summary component the same as or similar to summary component 22 (shown in FIG. 1 and described herein).

At an operation 306, presentation of the activity summary is effectuated on a user interface of a client computing platform. In some embodiments, operation 306 is performed by a presentation component the same as or similar to presentation component 23 (shown in FIG. 1 and described herein).

At an operation 308, a prompt is determined for an interaction between the subject and the caregiver. The prompt is based on the activity information. In some embodiments, operation 308 is performed by a prompt component the same as or similar to prompt component 24 (shown in FIG. 1 and described herein).

At an operation 310, presentation of the prompt is effectuated on the user interface of the client computing platform. In some embodiments, operation 310 is performed by a presentation component the same as or similar to presentation component 23 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A system configured to provide prompts to improve communication between users, the users including a subject and a caregiver, the system comprising:
one or more physical processors configured via computer-readable instructions to:
obtain activity information characterizing one or more activities involving a subject, wherein the activity information conveys one or both of a physical activity level for one or more time frames and/or calendar information for one or more time frames;
generate an activity summary of the activity information;
effectuate presentation of the activity summary on a user interface of a client computing platform that is associated with the caregiver;
determine a prompt for an interaction between the subject and the caregiver, wherein the prompt is based on the activity information; and
effectuate presentation of the prompt on the user interface of the client computing platform.

2. The system of claim 1, wherein the one or more physical processors are further configured to:
determine a quality metric based on the obtained information, wherein the quality metric reflects one or both of a level of reciprocity of a set of interactions between the subject and the caregiver and/or a frequency of interactions between the subject and the caregiver,
wherein the prompt is further based on the quality metric.

3. The system of claim 1, wherein the one or more physical processors are further configured to:
determine a set of activities that reflects one or both of an average day of the subject and/or an average week of the subject; and
determine whether a particular activity in the obtained information is unusual compared to the determined set of activities,
wherein the prompt is further based on the comparison

4. The system of claim 1, wherein the one or more physical processors are further configured to:
determining a first rate of interactions between the subject and the caregiver during a first time frame spanning at least a week;
determine a second rate of interactions between the subject and the caregiver during a second time frame, wherein the second time frame is on average more recent than the first time frame;
determine a difference between the first rate and the second rate; and
compare the difference to a differential rate threshold,
wherein the prompt is further based on the comparison.

5. The system of claim 1, wherein the activity information is obtained from a sensor configured to generate output signals conveying information related to the physical activity level of the subject, wherein the one or more physical processors are further configured to:
obtain a second set of activity information characterizing one or more activities involving the caregiver, wherein the second set of activity information conveys calendar information of the caregiver for one or more time frames;
generate a second activity summary of the second set of activity information;
effectuate presentations of the second activity summary on a second user interface of a second client computing platform that is associated with the subject;
determine a second prompt for a second interaction between the subject and the caregiver, wherein the second prompt is based on the second set of activity information; and
effectuate presentation of the second prompt on the second user interface of the second client computing platform.

6. A method of providing prompts to improve communication between users, the users including a subject and a caregiver, the method being implemented in a computer system that includes one or more physical processors and physical storage, the method comprising;
obtaining activity information characterizing one or more activities involving a subject, wherein the activity information conveys one or both of a physical activity level for one or more time frames and/or calendar information for one or more time frames;
generating an activity summary of the activity information;
effectuating presentation of the activity summary on a user interface of a client computing platform;
determining a prompt for an interaction between the subject and the caregiver, wherein the prompt is based on the activity information; and
effectuating presentation of the prompt on the user interface of the client computing platform.

7. The method of claim 6, further comprising:
determining a quality metric based on the obtained information, wherein the quality metric reflects one or both of a level of reciprocity of a set of interactions between the subject and the caregiver and/or a frequency of interactions between the subject and the caregiver;
wherein the prompt is further based on the quality metric.

8. The method of claim 6, further comprising:
determining a set of activities that reflects one or both of an average day of the subject and/or an average week of the subject;
determining whether a particular activity in the obtained information is unusual compared to the determined set of activities;
wherein the prompt is further based on the comparison.

9. The method of claim 6, further comprising:
determining a first rate of interactions between the subject and the caregiver during a first time frame spanning at least a week;
determining a second rate of interactions between the subject and the caregiver during a second time frame, wherein the second time frame is on average more recent than the first time frame;
determining a difference between the first rate and the second rate; and
comparing the difference to a differential rate threshold;
wherein the prompt is further based on the comparison.

10. The method of claim 6, wherein obtaining the activity information includes generating output signals conveying information related to the physical activity level of the subject, the method further comprising:
obtaining a second set of activity information characterizing one or more activities involving the caregiver, wherein the second set of activity information conveys calendar information of the caregiver for one or more time frames;
generating a second activity summary of the second set of activity information;
effectuating presentations of the second activity summary on a second user interface of a second client computing platform that is associated with the subject;
determining a second prompt for a second interaction between the subject and the caregiver, wherein the second prompt is based on the second set of activity information; and
effectuating presentation of the second prompt on the second user interface of the second client computing platform.

11. A system configured to provide prompts to improve communication between users, the users including a subject and a caregiver, the system comprising:
means for obtaining activity information characterizing one or more activities involving a subject, wherein the activity information conveys one or both of a physical activity level for one or more time frames and/or calendar information for one or more time frames;
means for generating an activity summary of the activity information;
means for effectuating presentation of the activity summary on a user interface of a client computing platform that is associated with the caregiver;
means for determining a prompt for an interaction between the subject and the caregiver, wherein the prompt is based on the activity information; and
means for effectuating presentation of the prompt on the user interface of the client computing platform.

12. The system of claim 11, further comprising:
means for determining a quality metric based on the obtained information, wherein the quality metric reflects one or both of a level of reciprocity of a set of interactions between the subject and the caregiver and/or a frequency of interactions between the subject and the caregiver,
wherein operation of the means for determining the prompt is further based on the quality metric.

13. The system of claim 11, further comprising:
means for determining a set of activities that reflects one or both of an average day of the subject and/or an average week of the subject; and
means for determining whether a particular activity in the obtained information is unusual compared to the determined set of activities,
wherein operation of the means for determining the prompt is further based on the comparison.

14. The system of claim 11, further comprising:
means for determining a first rate of interactions between the subject and the caregiver during a first time frame spanning at least a week;
means for determining a second rate of interactions between the subject and the caregiver during a second time frame, wherein the second time frame is on average more recent than the first time frame;
means for determining a difference between the first rate and the second rate; and
means for comparing the difference to a differential rate threshold;
wherein operation of the means for determining the prompt is further based on the comparison.

15. The system of claim 11, wherein the means for obtaining activity information includes a sensor configured to generate output signals conveying information related to the physical activity level of the subject, the system further comprising:
means for obtaining a second set of activity information characterizing one or more activities involving the caregiver, wherein the second set of activity information conveys calendar information of the caregiver for one or more time frames;
means for generating a second activity summary of the second set of activity information;
means for effectuating presentations of the second activity summary on a second user interface of a second client computing platform that is associated with the subject;
means for determining a second prompt for a second interaction between the subject and the caregiver, wherein the second prompt is based on the second set of activity information; and
means for effectuating presentation of the second prompt on the second user interface of the second client computing platform.
